(19) United States
(12) Patent Application Publication
(10) Pub. No.: US 2022/0233559 A1
(43) Pub. Date: Jul. 28, 2022

(54) XYLOOLIGOSACCHARIDE AS A MULTIFUNCTIONAL PREBIOTIC

(71) Applicant: Prenexus Health, Inc., Gilbert, AZ (US)

(72) Inventor: Kevin A. Gray, San Diego, CA (US)

(73) Assignee: Prenexus Health, Inc., Gilbert, AZ (US)

(21) Appl. No.: 17/586,381

(22) Filed: Jan. 27, 2022

Related U.S. Application Data
(60) Provisional application No. 63/142,148, filed on Jan. 27, 2021.

(51) Int. Cl. A61K 31/702; A61P 1/14; A61K 9/00

(52) U.S. Cl. CPC A61K 31/702; A61K 9/0053; A61P 1/14

(57) ABSTRACT

This disclosure relates to the co-production and co-delivery of a pre- and postbiotic and antioxidant that are chemically linked and thus can be delivered as a single product instead of a blend of individual molecules. This xylooligosaccharide (XOS) product is used as a nutritional supplement and/or food ingredient that improves digestive health and overall wellness.

This application claims the benefit of U.S. Provisional appl. No. 63/142,148, filed on Jan. 27, 2021, which is hereby incorporated by reference herein in its entirety.

### TECHNICAL FIELD

This invention relates to the co-production and co-delivery of a pre- and postbiotic and antioxidant that are chemically linked and thus can be delivered as a single product instead of a blend of individual molecules. This xylooligosaccharide (XOS) product is used as a nutritional supplement and/or food ingredient that improves digestive health and overall wellness.

### BACKGROUND

Co-delivery of nutritional supplements and vitamins is a challenging enterprise. Oftentimes nutritional and dietary supplements are formulated such that one or more active components can be delivered to a subject, either simultaneously or stepwise.

However, as more components are added to the formulation, it becomes more difficult with respect to delivery to the subject and chemical stability.

If a way could be found to combine both production and delivery of active materials comprising a prebiotic, a postbiotic and an antioxidant in a single product, this would serve as a useful contribution to nutrition science.

Xylo-oligosaccharides are a nutrient (carbon) source for beneficial anaerobic micro-organisms in the digestive tract of the host. These microbes in turn produce metabolites that are beneficial to the host which then provide a physiological benefit to the host. Microbial metabolites include, but are not limited to, short chain fatty acids (SCFA) including acetic, butyric, propionic, etc acids. Due to providing nutrition to gut beneficial microbes xylooligosaccharides are referred to as “prebiotics”. See, Saville, B. A. and Saville, S., Appl. Food Biotech. (2018) 5(3): 121-130; Saville, S. and Saville, B. A., Agro Food Industry Hi Tech (November/December 2018) 29(6): 36-38.

If a way could be found to use xylooligosaccharides to deliver other needed nutrients to an organism, such as vitamins or antioxidants, this would be considered a contribution to the nutritional and medical arts.

### SUMMARY

In an embodiment, a method is described for providing a prebiotic, a postbiotic, and an antioxidant, to a host organism, comprising the steps of: (a) providing a xylo-oligosaccharide product having formula (I), and (b) administering the xylo-oligosaccharide to the host in an amount effective to deliver acetic acid and/or a cinnamic acid derivative after enzymatic cleavage

### BRIEF DESCRIPTION OF THE FIGURES

FIG. 1 depicts UPLC/ELSD/MS-TIC spectra of PreneXOS™. Top trace: ELSD chromatogram; bottom trace: ES-TIC spectrum. The sample shows an XOS degree of polymerization (DP) of approx. 2 to >10 (10+). In one useful embodiment, the xylooligosaccharide mixture has a degree of polymerization “DP” of about 3 to 12. In another useful embodiment, the DP range can include greater than DP12, i.e. 12+.

FIG. 2 depicts ES mass spectra of PreneXOS™ showing an acetylated xylo-oligomer pattern (DP4-DP7).

FIG. 3 depicts, in an in vitro gastric model of the colon (TIM-2), the effects of three doses of PreneXOS™ at 1.0 g/day, 1.5 g/day, and 3.0 g/day compared to control medium (SIEM). Cumulative production of short chain fatty acids (SCFAs) is expressed as SCFA produced per gram of XOS provided per day. Acetate: solid squares; propionate: triangles; butyrate: X's. X-axis is hours: 24 h, 48 h, 72 h.

### DETAILED DESCRIPTION

In its principal embodiment, the invention comprises a xylooligosaccharide (PRENEXOS™, available from Prenexus Health, Inc., Gilbert, Ariz.), a mixture of xylo-oligosaccharides of various chain lengths (a.k.a. PreneXOS™, Prenexus XOS™, and XOS95®). In one useful embodiment, the xylooligosaccharide mixture has a degree of polymerization “DP” of about 3 to 12, based on xylose monomers. In another useful embodiment, the DP range can include greater than DP12, i.e. 12+, based on xylose monomers.

In a preferred embodiment, PRENEXOS™ is derived from sugar cane using a process that has been certified organic and includes no chemical addition except for water. In other embodiments, XOS may be prepared using other plant-based materials or feedstocks, such as corn cobs or wheat straw, for example.

PRENEXOS™ is not a simple straight chain XOS but also contains branches of sugar residues, and substitution by acetate and polyphenolics as esters, therefore achieving co-delivery of the short chain fatty acid (SCFA) acetic acid, and also antioxidant polyphenolic compounds such as ferulic acid, p-coumaric acid, 3,4-dihydroxycinnamic acid, and 3,5-dimethoxy-4-hydroxycinnamic acid.

A representative structure of PRENEXOS™ is shown in Formula (I):

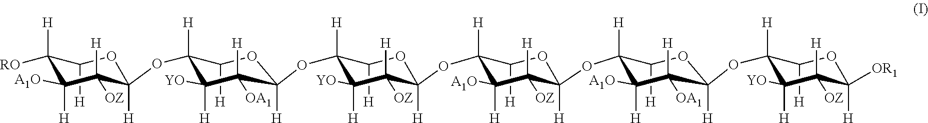

wherein R and R₁ are each independently selected from hydrogen or one or more xylose units. In an embodiment, the xylose are (beta-1,4-xylose, or beta-1,4-xylosyl) residues. Other configurations of the polysaccharide are contemplated. Xylose units may be removed from Formula (I) when it occurs in a mixture of oligosaccharides, and merely substituted with a hydrogen atom.

In Formula (I), A₁ is selected from hydrogen or acetyl.

In Formula (I), Y is selected from hydrogen, arabinose (arabinosyl), galactose (galactosyl), ribose (ribosyl), mannose (mannosyl), glucuronic acid (glucuronosyl), or glucose (glucosyl), and the like, in any of several linkage configurations (taking into account mutarotation and steric effects). Z is selected from hydrogen, glucuronic acid (glucuronosyl), galacturonic acid (galacturonosyl), mannuronic acid (mannuronosyl), and the like, or methylated or alkylated derivatives thereof, in any of several linkage configurations.

In an embodiment, Y and Z can be exchanged one for another. For example, instead of —OY at the 4-carbon position of xylose, it may be at the 3-carbon position, and vice versa. For example, instead of —OZ at the 3-carbon position of xylose, it may be at the 4-carbon position, and vice versa.

In an embodiment, either of Y or Z may be further derivatized (i.e., substituted on a sugar hydroxyl) as a cinnamate ester, or derivatized or substituted cinnamate ester (i.e., cinnamoyl substitution of a sugar hydroxyl). In an embodiment, the phenolic side chains may comprise other cinnamate type structures. For example, if the positions of the phenyl group -meta, -para, -meta are considered in that order as R₁₁, R₁₂, and R₁₃ respectively, then each of R₁₁, R₁₂, and R₁₃ are each independently selected from hydrogen, hydroxyl (—OH), or methoxy (—OCH₃).

It may be understood that the XOS materials described herein is based on a hemicellulose fraction and these materials comprise a mixture of compounds having varying chain lengths in terms or the sugar, i.e. saccharide backbone, most of the sugar residues being xylose in xylo-oligosaccharide. Thus, as used herein the terms “oligosaccharide”, “oligosaccharides”, and “XOS” may be construed as referring to a compound or group of compounds having varying chain lengths and branch points in the carbohydrate backbone.

In one embodiment, a representative structure of PRENEXOS™ is shown in Formula (Ia):

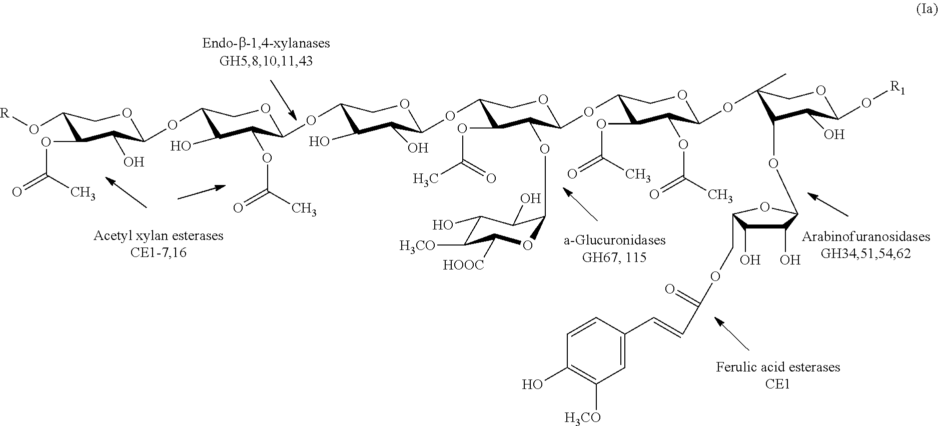

wherein R and R₁ are each independently selected from hydrogen or one or more xylose units.

It should be understood that an oligosaccharide is by definition a material comprising a mixture of compounds having varying chain lengths in terms or the sugar, i.e. saccharide backbone, most of the sugar residues being xylose in xylo-oligosaccharide. Thus, as used herein the terms “oligosaccharide”, “oligosaccharides”, and “XOS” may be construed as referring to a compound or group of compounds having varying chain lengths and branch points in the carbohydrate backbone.

In an embodiment, the phenolic side chains may comprise other cinnamate type structures. For example, if the positions of the phenyl group -meta, -para, -meta are considered in that order as R₁₁, R₁₂, and R₁₃ respectively, then each of R₁₁, R₁₂, and R₁₃ are each independently selected from hydrogen, hydroxyl (—OH), or methoxy (—OCH₃).

Table 1 shoes the characterization of a representative sample of PRENEXOS™ having Formula (I).

Characterization of XOS

TABLE 1

| | Specifi- | Measured | |
|---|---|---|---|
| Component | cation | Amount | Analysis Method |
| Appearance | Powder | Powder | Visual |
| Color | Off-white | Off-white | Visual, ICUMSA |
| | to light | | |
| | tan | | |
| Total solids, wt % | >93 | 97.7 | AOAC 925.45 |
| Water activity | | 0.11 | ISO 18787:2017 |
| Xylooligosaccharides, | >75 | 85 | NREL/TP-510-42618 |
| wt % | | | (HPLC) |
| Carbohydrate | | | NREL/TP-510-42618 |
| Monomers | | | (HPLC) |
| Glucose/Fructose/ | <12 | 0 | NREL/TP-510-42618 |
| Sucrose/Xylose, wt % | | | (HPLC) |
| Polyphenols and | <3.0 | 0.57 | AOAC 986.13 and |
| organic acids, wt % | | | Methods of Enzymology, |
| | | | 299, pp 152-178 (1999) |

Batch number: XOS-211104

LC-MS analysis carried out by the National Renewable Energy Laboratory in Golden, Colo. according to Xiong, W., Reyes, L. H., Michener, W. E., and Maness, P.-C., Engineering cellulolytic bacterium Clostridium thermocellum to co-ferment cellulose- and hemicecellulose-derived sugars simultaneously, Biotechnology and Bioengineering (2018) 115 (7), 1755-1763.

Degree of Polymerization and Acetylation

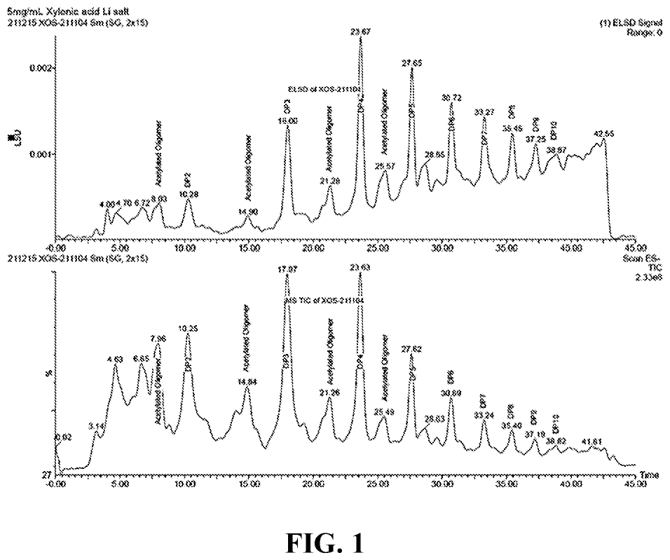

FIG. 1 shows UPLC/ELSD/MS-TIC chromatogram & spectra of PreneXOS™. The sample shows an XOS degree of polymerization (DP) of approx. 2 to >10 (10+). The peaks eluting between the XOS chains are identified as acetylated xylo-oligomers (i.e., A₁ is acetyl in Formula (I)).

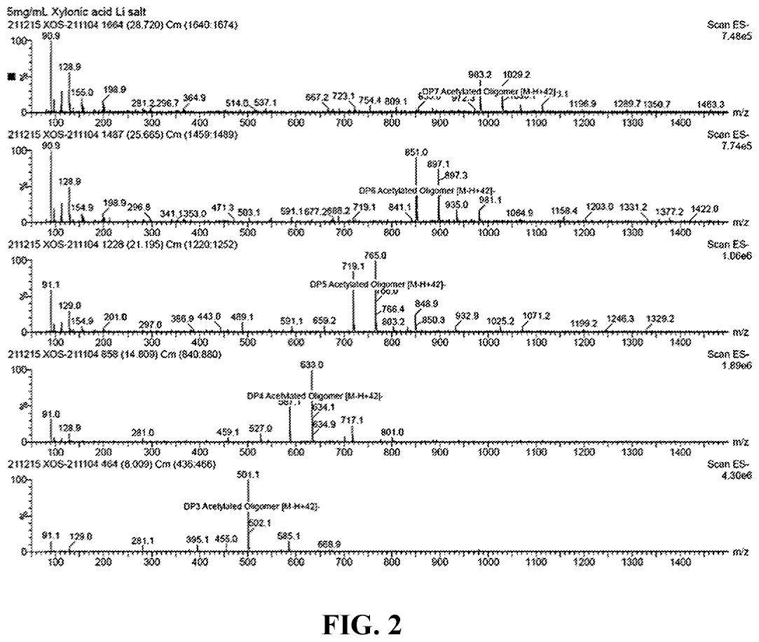

FIG. 2 show the extracted mass spectra of some of the peaks eluting between the xylo-oligomers (DP4-DP7) of XOS-211104 show an acetylated xylo-oligomer pattern.

Chemical Analysis of Acetylation and Non-Xylose Sugar Branches

Table 2 shows the mol/mol ratios of acetate and certain sugar branches including galactose (galactosyl) and arabinose (arabinosyl) which are present in materials having Formula (I).

TABLE 2

| XOS Substitution/ | Xylose/ | Xylose/ | Xylose/ |
|---|---|---|---|
| Branching | acetate | galactose | arabinose |
| Average (mol/mol) | 5.9 | 38.2 | 28.5 |
| Standard deviation | 1.4 | 6.0 | 8.8 |

Based on Table 2, on average, every 6^{th} xylose in PreneXOS™ is acetylated while every 40^{th} appears to have a galactose side branch and every 30^{th} appears to have an arabinose side branch, when the material is taken as a whole, in any given sample.

Due to providing nutrition to gut microbes xylooligosaccharides are referred to as “prebiotics”. As stated above, xylooligosaccharides are a nutrient (carbon) source for beneficial anaerobic micro-organisms in the digestive tract of the host. These microbes in turn produce metabolites that are beneficial to the host which then provide a physiological benefit to the host. Microbial metabolites include, but are not limited to, short chain fatty acids (SCFA) including acetic, butyric, propionic, etc acids. SCFA are sometimes referred to as “postbiotics” since they themselves are one of direct sources of the physiological effect on the host. Another class of compounds such as polyphenolics are referred to as “antioxidants” since they “scavenge” reactive oxygen species that are detrimental. Antioxidants also provide a beneficial effect to the host. PRENEXOS™ is composed of a backbone of xylose monomers linked together but also containing acetyl esters and ferulic acid esters. All three components are produced together in a single unit as opposed to separately manufactured.

Thus, in another embodiment, the invention relates to the co-production and co-delivery of a prebiotic, a postbiotic and an antioxidant that are chemically linked and thus can be delivered as a single product instead of a blend of individual molecules. The product, PRENEXOS™, is used as a nutritional supplement and/or food ingredient that improves digestive health and overall wellness.

Xylooligosaccharides are derivatives of the hemicellulose fraction found in plant material. Hemicellulose is a complex structural polysaccharide that, in certain plants like sugar cane, has a xylan backbone with branches of other sugars such as arabinose, galactose, mannose, glucuronic acid and sometimes glucose. In addition to the sugar branches hemicellulose is connected to acetyl (i.e. acetylated), ferulic and diferulic acids and p-coumaroyl that link xylan chains to lignin. Sugar cane has hydroxycinnamic acid (ferulic, coumaric and sinapic acids) involved in crosslinking xylan and lignin molecules. During Prenexus Health's (i.e., the applicant's) high temperature “cook” process that produces PRENEXOS™, acetyl esters are hydrolyzed releasing acetic acid into solution decreasing the pH. The low pH and high temperature then catalyzes the hydrolysis of glycosidic linkages between xylose subunits in the xylan chain resulting in shorter chain, water soluble xylo-oligosaccharides. However, the process conditions are not severe enough to completely debranch XOS therefore some acetyl and ferulic acid esters remain as do branches of other sugars and various polyphenolics and lignin fragments. The present application uses a series of filtration steps (micro-, nano- and ultra-) along with ion exchange chromatography to isolate XOS from the crude extract. While ion exchange chromatography is effective at removing color bodies we observed an increase in free acetic acid after use of the resin when run under alkaline conditions. Mass balance studies showed that the ion exchange process catalyzed the conversion of bound acetate to unbound acetic acid. Even though the ion exchange process catalyzes the partial de-esterification of acetyl groups some still remain covalently bound to the XOS backbone. Furthermore, some color still remains bound to the XOS background which is an indicator of polyphenolics being present.

In addition to the xylose backbone, the XOS may contain glucuronic acids, connected at the 2-position of xylose, and arabinose at the 3-position which may be esterified as shown in Formula (I).

According to F. Shahidi and J. Yeo, Insoluble-Bound phenolics in food, Molecules (2016) 21:1216, in plant cell walls “phenolic compounds can form covalent bonds with cell wall substances such as cellulose, hemicellulose, arabinoxylans, structural proteins and pectin through ester, ether and C—C bonds. The carboxyl group of phenolic acids such as benzoic and cinnamic acids can form ester bonds with hydroxyl groups of cell wall substances and C—C bonds as well when they directly create covalent bond between carbon atoms of phenolics and carbon atom of cell wall substances.”

Chemical analysis of PRENEXOS™ revealed the presence of bound phenolic groups (in addition to acetate groups). A standard assay is performed to detect the presence of polyphenolics that involves a methanol extraction and then a colorimetric assay. Methanol will dissolve “free” phenolic groups in solution and therefore does not detect methanol insoluble polyphenolics (i.e. those bound to XOS); if we use water as the solvent we measure a much higher phenolic content and our interpretation of those data is that water dissolves all components (including XOS) and therefore measures bound and free.

Table 3 shows the results of the results from the polyphenolic assay for one batch of product produced in the manufacturing plant.

TABLE 3

| | Total phenolic content | Free phenolic content |
|---|---|---|
| Sample | (% solids) | (% solids) |
| XOS200918 | 0.91 | 0.04 |

The difference between “total phenolic content” and “free phenolic content” is that which is bound to XOS. Per observation, “total phenolic content” is measured by dissolving XOS into water and carrying out the assay whereas “free phenolic content” is measured by extracting powdered XOS with methanol (XOS is insoluble in MeOH whereas phenols are soluble in MeOH) and carrying out the assay on the MeOH fraction. The numbers are wt percent of the solids.

Thus, one disadvantage of using PRENEXOS™, manufactured in this way is the presence of color. The final product is not a white to off-white product but yellow to tan. The yellow color of PRENEXOS™ is pH dependent. Under alkaline conditions a solution of PRENEXOS™ in water is yellow whereas under neutral to acidic conditions the solution is colorless.

In contrast, the advantage of the XOS manufacturing process is that the process enables production of XOS containing both acetyl substitution and phenolic or cinammic ester branching. Each of these types of side chains, once cleaved in situ, or in vivo, may be delivered to an organism or subject in the form of a short chain fatty acid (acetic acid, in this case), or a cinnamic acid derivative (such as, ferulic acid). It is evident from this approach that other SCFAs may be delivered, or other phenolic/cinnamic acid compounds may be delivered, in accordance with the principles of the invention herein.

The advantage of this system is that PRENEXOS™ combines three properties into a single molecule; (1) a prebiotic effect from the XOS backbone providing nutrition for beneficial microorganisms, (2) a postbiotic effect by delivering acetic acid directly to the digestive tract, and (3) anti-oxidant activity by delivering phenolic compounds along with XOS. No additional cost or time is required to produce the combined molecule and no blending is required. In an embodiment, the XOS product may be administered orally to a human subject in a daily dose of about 1 g.

In the host or subject the XOS backbone is digested enzymatically to produce “fermentable sugars”, those sugars are then metabolized by beneficial gut microbes thereby producing short chain fatty acids (SCFA) and other metabolites. See, formula (I) above. The SCFA and other metabolites then elicit a beneficial health response in the host. PRENEXOS™ has the added benefit of being a “carrier” for additional SCFA (mainly acetic acid) thereby improving performance relative to a non-branched XOS. In addition, PRENEXOS™ is also a “carrier” for phenolic groups that are released enzymatically and add antioxidant activity to the use of PRENEXOS™.

This is unusual and unexpected since other methods to produce XOS describe methods that would result in a “straight chain” molecule devoid of any branches. The branches will aid performance by adding the additional activities.

The advantage of this invention may be described as follows. Insoluble-bound phenolics are not absorbed in the small intestine because they are bound to insoluble macromolecules such as cellulose, hemicellulose, structural protein and pectin. Thus they reach the colon (large intestine), where they undergo fermentation by the colon microbiota and release the bound phenolics. In the colon, a variety of microorganisms, exist and take part in the fermentation of the unadsorbed material. The microorganisms, including Bifidobacterium spp. and Lactobacillus spp. secrete a variety of extracellular enzymes such as carbohydrases, proteases and other types of enzymes, leading to the disruption of cell wall matrix or hydrolysis of covalent bonds of bound phenolics, followed by liberation of phenolics. The released phenolics render a myriad of health benefits (Shahidi and Yeo, 2016).

Raw materials other than sugar cane can be used to produce a similar molecule. Grassy feedstocks such as wheat and rice straw, corn stover, Miscanthus, switch grass, elephant grass, Arundo donax, sorghum, etc could also be used. Woody feedstocks would be expected to produce a different molecule due to the presence of mannose in the hemicellulose backbone as opposed to xylose.

Alternative process technologies (“pretreatment”) could also be used to produce a similar molecule. This disclosure currently utilizes “autohydrolysis” in a slurry vessel for the thermochemical hydrolysis, whereas dilute acid hydrolysis and/or alternative reactor designs (for example plug flow) could also be used.

### Example 1

A study was performed using an in vitro model of the colon (“TIM-2”) to simulate human adults.

The results are reported in K. Venema, et al., “Xylo-oligosaccharides from sugarcane show prebiotic potential in a dynamic computer-controlled in vitro model of the adult human large intestine”, Beneficial Microbes (2020) 11(2): 191-200, which is herein incorporated by reference in its entirety for all purposes, along with the references cited therein.

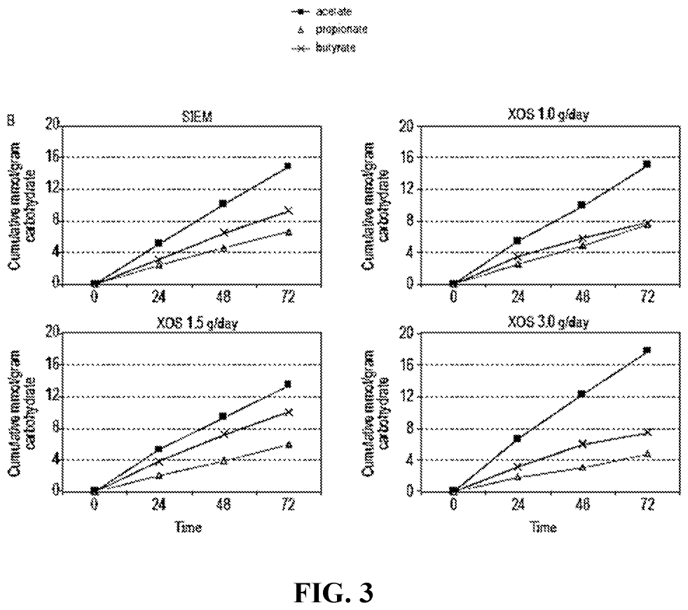

The effects of three doses of PreneXOS™ at 1.0 g/day, 1.5 g/day, and 3.0 g/day were compared to control medium (SIEM). As shown in FIG. 3, acetate increased in a dose dependent manner at the 3.0 g dose.

Dynamic Gastro-Intestinal (GI) Models

The TNO (the Dutch Organisation for Applied Life Sciences) in vitro gastrointestinal models simulate to a high degree the successive dynamic processes in the stomach, the small intestine (“TIM-1”, Minekus et al., 1995, A multi compartmental dynamic computer-controlled model simulating the stomach and small intestine, Alternatives to Laboratory Animals (ATLA) 23: 197-209; Havenaar and Minekus, 1996, Simulated assimilation. Dairy Industries International 61 (9): 17-23) and in the large intestine (“TIM-2”, See FIG. 1; Minekus et al., 1999, A computer-controlled system to simulate conditions of the large intestine with peristaltic mixing, water absorption and absorption of fermentation products, Appl. Microb. Biotechn. 53: 108-114; Venema et al., 2000, TNO's in vitro large intestinal model: an excellent screening tool for functional food and pharmaceutical research. Ernährung/Nutrition 24 (12): 558-564). During the experiments samples from different sites of the GI tract can be taken in time. The aforementioned references are hereby incorporated by reference.

As used herein, the term “TIM-2” refers to TNO's in vitro gastrointestinal model of the large intestine (colon), as described herein.

These models are unique tools to study the stability, release, dissolution, absorption and bioconversion of nutrients, chemicals, bioactive compounds and pharmaceuticals in the gastrointestinal tract. This provides insight in the (rate of) digestibility and kinetics of bioaccessibility of nutrients and/or the stability and activity of functional ingredients, such as probiotics in the upper GI tract, and the effect of functional ingredients, such as prebiotics, on the composition and activity of the gut microbiota in the colon.

### Example 2

Clinical Study of Prebiotic Effects of Xylo-Oligosaccharides (PRENEXOS™) on Microbiome Modulation and GI Tolerance

Purpose

Xylo-oligosaccharides (XOS Prebiotic) are a type of prebiotic that selectively stimulate the growth of Bifidobacteria and Lactobacilli, which can increase the production of short chain fatty acids (SCFAs) and lower gut pH. The purpose of this study was to examine the effects of oral supplementation with PRENEXOS™, a xylo-oligosaccharide prebiotic manufactured by Prenexus Health, Inc., on microbiome modulation and gastrointestinal tolerance.

Further, it was hypothesized that oral supplementation with 1,000 mg/day of PRENEXOS™ would increase intestinal contents of Bifidobacteria and SCFAs while promoting shifts in microbiome microbial populations.

Methods

Using an open-label design, 12 healthy volunteers (44.3±7.8 years, 77.9±19.0 kg and 25.6±4.7 kg/m2) were assigned to ingest one dose (two capsules) before noon each day, with each capsule containing 500 mg PRENEXOS™. At baseline, week 2, week 4, and week 6 the following assessments were made: body weight, vital signs (heart rate, blood pressure), stool frequency questionnaire, visual analog scales for flatulence, bloating, rumbling, cramps, nausea, & stool consistency, total bacterial plate counts, short chain fatty acid (SCFA) and 16s rRNA, and adverse event monitoring.

Summary of Results

No clinically meaningful changes occurred in body mass, blood pressure, or resting heart rate over the course of the study. SCFA levels were unchanged in practically all subjects. In response to supplementation, robust changes in Bifidobacteria were identified while changes in Lactobacillus were considered moderate. Analyses of data for flatulence and bloating revealed consistent increases while gastrointestinal cramping decreased in both genders with no differences between gender across time. The increases in gastrointestinal rumbling were significantly greater in men vs. women. No clinically significant changes were noted for any of the whole blood or clinical safety markers assessed.

Materials and Methods

This study was completed as an open label pilot study that examined the effects of six weeks of PRENEXOS™ supplementation on changes in gut microbiota, adverse events, clinical safety, and other indicators of stool production and quality. All procedures involving human subjects were approved (Protocol #PRENEX-001-2019, Approval date: Feb. 12, 2019) by the Institutional Review Board at Integreview, Inc. (Austin, Tex.). Written consent was obtained from all participants prior to any participation.

Participants

Twelve apparently healthy subjects between the ages of 30 and 60 years old participated in this study. All subjects were pre-screened using health history questionnaires, vital signs, and blood work. Participants were required be: of a normal weight (body mass index between 20-35 kg/m²), normotensive (systolic blood pressure <140 mm Hg and diastolic blood pressure <90 mm Hg), recreationally active (at least 3 days/week), and report having normal bowel habits (defined as between 1-3 stools per day). Subjects were excluded if they were: using any nutritional supplement known to alter the gut microbiome/microflora in the previous four weeks and for the duration of the study, using any antibiotics, antifungals, antivirals, or antiparasitics within six months of the start of the study. Finally, participants were excluded if they had a gastrointestinal or metabolic disease that might impact nutrient absorption, inborn errors of metabolism, or had any chronic inflammatory condition or disease. Each subject's baseline diet (24-hour diet record) was analyzed to determine its energy and macronutrient content and to ensure consistent dietary fiber intake of 20-30 grams per day from foods. Subjects were required to maintain stable fiber intake in conjunction with making no purposeful changes to their diet throughout the duration of the study. In addition, and to replicate baseline-testing conditions as closely as possible, subjects were given a copy of dietary records they completed at the start of the study and were asked to replicate their dietary pattern for the 24 hours prior to each testing visit in addition to observing a 10-hour fast prior to each laboratory visit.

Experimental Protocol

A summary of the study design is presented in Table 4. All testing was performed at the Center for Applied Health Sciences. Interested participants responded to advertisements regarding study participants and completed an initial eligibility screening over the phone. Participants meeting initial eligibility were scheduled for a screening visit where they completed a health history, signed a consent, had vital signs and demographics assessed, and provided a fasting venous blood sample for determination of clinical safety markers. Within seven days, participants were scheduled for their initial study visit where they had vital signs and demographics assessed, provided a fecal sample, dietary record, physical activity record, and were provided the test product in an open label fashion. Participants were scheduled for subsequent study visit every two weeks (2, 4, and 6 weeks after the start of supplementation) and completed a battery of tests that were identical in sequence to the baseline study visit (week 0).

TABLE 4

| | | Visit 2 | Visit 3 | Visit 4 | Visit 5 |
|---|---|---|---|---|---|
| PROCEDURE | Screening | (Week 0) | (Week 2) | (Week 4) | (Week 6) |
| Informed Consent | X | | | | |
| Inclusion/Exclusion Criteria | X | | | | |
| Medical History | X | | | | |
| Demographics & Vitals | X | X | X | X | X |
| Venous Blood Safety Screen | X | | | | X |
| Total Bacterial plate | | X | X | X | X |
| counts/Culture and KITS | | | | | |
| SCFA and 16s rRNA | | X | X | X | X |
| Kit for SCFA and 16s rRNA | | X | X | X | X |
| Stool frequency | X | X | X | X | X |
| Gastrointestinal Health VAS | X | X | X | X | X |
| Dietary Analysis | X | | X | X | X |
| Physical Activity | | X | X | X | X |
| Dispense Test Prouduct | | X | | | |
| Adverse Event Monitoring | | X | X | X | X |

Fecal Collection

Study participants were provided all necessary supplies and instructions to collect a small fecal sample into a sterile vessel at approximately the same time each day. To reduce sample contamination, participants were told to urinate before collecting the stool sample and were advised to not have taken any antacids, barium bismuth, or anti-diarrhea medications/laxatives prior to collection. Stool samples were collected in this manner at baseline and after two, four, and six weeks of supplementation. In each sample, bacterial plate counts, SCFA analysis, and 16S rRNA sequencing were performed on frozen (−800 C) stool samples by a commercial laboratory.

Gastrointestinal Health Ratings

Prior to and every two weeks after supplementation, study participants were asked to record questions about their stool frequency, appearance, and consistency. In addition, visual analog scales (VAS) were completed by study participants at baseline and after two, four, and six weeks of supplementation. All VAS were constructed similarly with a 100-mm line anchored by “Lowest Possible” and “Highest Possible” to assess subjective ratings of flatulence, intestinal bloating, intestinal rumbling, intestinal cramping, nausea, stool consistency, and urgency to empty bowels. The validity and reliability of VAS to assess various psychosomatic and clinical indicators has been previously established.

Statistical Analysis

Statistical analyses were performed using SPSS V.25 (IBM Corporation; Armonk, N.Y.). Primary and secondary outcome measures were initially analyzed by a 2×2 (gender×time) repeated measures analysis of variance (ANOVA). Normality was assessed using the Shapiro-Wilk test. Non-normal variables were transformed using log 10 or square root transformations before completing ANOVA procedures. Raw data are reported throughout tables and figures. Homogeneity of variance was assessed using Mauchly's Test of Sphericity. When the homogeneity of variance assumption was violated, the Greenhouse-Geisser correction was applied when epsilon was <0.75 and the Huynh-Feldt correction was applied when epsilon was >0.75. Bonferroni confidence interval adjustments were made to evaluate specific changes across time. A statistically significant finding was determined as a p-value of less than or equal to (p≤0.05) while a trend was defined as a p-value of 0.06-0.10. Effect sizes (ES) were also calculated to determine the magnitude of treatment effects.

Supplementation

In an open-label fashion, study participants were assigned to ingest one dose before noon each day of the study protocol. Each dose consisted of two capsules providing a total of 1,000 mg of PRENEXOS™ (500 mg each capsule) per dose. As part of compliance monitoring to the supplementation regimen, participants were provided the capsules on a weekly basis in a pill container that provided seven doses and were asked to return the empty containers. A side effects questionnaire was also completed when receiving capsules. Compliance was set at ≥80%, and participants not meeting compliance were removed from the study.

Results

Demographics, Hemodynamics, and Physical Activity

Twelve healthy adults (5 males, 7 females) completed the study protocol. Participant demographics and clinical vital indicators are provided in table 1. Body mass, systolic blood pressure, and diastolic blood pressure experienced no statistically significant changes across time or between gender (p>0.05) while resting heart rate experienced a statistically significant (p<0.05) increase (approximate 4-6 beats per minute) in both genders after two weeks of supplementation which subsequently returned to reported baseline levels after eight weeks of supplementation. Physical activity ratings (Framingham score) experienced a statistically significant (p=0.05) gender×time interaction, with men increasing their reported level of physical throughout the six-week study protocol.

Fecal Bacterial Counts

No statistically significant changes were observed in the number of reported stool collections per day in either gender from the beginning to the end of the study protocol (p>0.05). As seen in Table 5, the content of Bifidobacteria present in collected fecal samples increased substantially in all study participants. Notably, one hyper-responder was present in the Bifidobacteria group, which led to the calculation of additional metrics to ensure a more tempered interpretation of the potential changes observed in the entire sample. Consequently, in Table 5 below, we have excluded the highest and lowest subject at each timepoint and have subsequently reported a “trimmed-mean”. A retrospective examination of patient demographic characteristics indicated that subjects who responded to treatment with a robust increase in Bifidobacteria were generally at least 40 years old and had a BMI>28. No gender specific responses were identified. Finally, calculated levels of short-chain fatty acids (SFCAs) were unchanged in practically all study participants.

In Table 5, Bifidobacteria number was reported as % change as compared to baseline.

TABLE 5

| | All subjects | | Hyper-Responder Removed | |
|---|---|---|---|---|
| | Reactive | CFU | Reactive | CFU |
| Week | Abundance | Counts | Abundance | Counts |
| 2 | 100% | 195% | 81% | 157% |
| 4 | 198% | 285% | 83% | 159% |
| 6 | 398% | 332% | 37% | 53% |

As seen in Table 6, the content of Lactobacillus present in collected fecal samples increased in what is described as a modest fashion. All observed changes in Lactobacillus were reasonable and expected changes in response to the study intervention. No gender specific responses were identified.

In Table 6, Lactobacillus number was reported as % change as compared to baseline.

TABLE 6

| | All subjects | |
|---|---|---|
| Week | Relative Abundance | CFU Counts |
| 2 | 21% | 43% |
| 4 | 2% | 21% |
| 5 | 36% | 35% |

Visual Analog Scales—Gastrointestinal Side Effects

Mixed factorial ANOVA were computed for all visual analog scales to assess changes in VAS ratings for flatulence, rumbling, cramping, nausea, stool consistency and urge to empty bowels. For all variables, no significant gender×time interaction (p>0.05) were observed and no significant main effects (p>0.05) for time were observed for both genders. A 15.5% increase (Effect Size [ES}, d=0.20) in flatulence was reported in both genders, but this change was non-significant (within-group p-value=0.55), nor were changes different between genders (gender×time, p=0.69). A 44.7% increase (ES, d=0.40) in GI bloating per day was reported in both genders, but this change was non-significant (within-group p-value=0.43), nor were changes different between genders (gender×time, p=0.54). A 22.2% increase (ES, d=0.20) in GI rumbling per day was reported in both genders, but this change was non-significant (within-group p-value=0.38). Males reported significantly greater indices of GI rumbling when compared to females, p=0.04). A 15.5% decrease (ES, d=0.12) in GI cramping per day was reported in both genders, but this change was non-significant (within-group p-value=0.27), nor were changes different between genders (gender×time, p=0.74). Moreover, the overall reported decrease resulted from week 4 to week 6, which can lead one to speculate further decreases could have occurred had supplementation continued for a longer period of time.

Further, all clinical safety outcomes were measured including blood and serum indicators.

### CONCLUSIONS

Supplementation was well tolerated with no clinically significant changes in adverse events, fecal content, or whole blood and serum indicators of safety. While changes in gastrointestinal ratings were observed, no meaningful differences were observed between genders. Finally, six weeks of supplementation with PRENEXOS™ resulted in robust increases in Bifidobacteria while moderate changes were observed for Lactobacillus, as shown herein.

This open label, pilot study investigation sought to determine the safety and efficacy of a six-week supplementation period of PRENEXOS™, a branded xylo-oligosaccharide prebiotic produced from high-fiber sugar cane by Prenexus Health, Inc. The primary findings of this pilot study revealed that supplementation was well tolerated with no clinically significant adverse outcomes and only marginal changes in perceived ratings of common gastrointestinal outcomes. Furthermore, a six-week supplementation period led to widespread and robust increases in Bifidobacteria and moderate increases in Lactobacillus.

In conclusion, results from this pilot study highlight preliminary effects associated with PRENEXOS™ prebiotic supplementation on changes in Bifidobacteria and Lactobacillus counts in collected fecal samples. Additionally, within these samples, no effect on the production of short-chain fatty acids were found nor were there changes in subjective markers of gastrointestinal function in normal, healthy men and women. Importantly, the supplementation protocol was well tolerated with reasonable and expected changes in reported gastrointestinal outcomes and no statistically significant changes in markers of clinical safety in the collected blood samples. The focus from this project should be on the development of future clinical research studies to more fully examine the potential safety and efficacy of PRENEXOS™. In this respect, future studies should employ larger sample sizes and utilize a placebo group so that normal variation in all endpoints can be determined. Additionally, based on our read of the scientific literature, the participants enrolled should have a body mass index of at least 27 kg/m² (classifying them as ‘overweight’) and should be at least 40 years old. Finally, to more precisely control dietary intake (which is a critical factor in studies measuring changes in microbiota), it is advised that participants are provided at least one meal and two snacks per day throughout the study protocol.

Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Other useful delivery systems comprise oral or nasal sprays or inhalers, and the like.

For oral administration, prebiotics may be further combined with one or more solid inactive ingredients for the preparation of tablets, capsules, pills, powders, granules, or other suitable dosage forms. For example, the active agent may be combined with at least one excipient selected from the group consisting of fillers, binders, humectants, disintegrating agents, solution retarders, absorption accelerators, wetting agents, absorbents, and lubricating agents. Other useful excipients include, but are not limited to, magnesium stearate, calcium stearate, mannitol, xylitol, sweeteners, starch, carboxymethylcellulose, microcrystalline cellulose, silica, gelatin, silicon dioxide, and the like.

In certain embodiments, the components of compositions administered according to the methods of the present disclosure, together with one or more conventional adjuvants, carriers, or diluents, may thus be placed into the form of pharmaceutical compositions and unit dosages thereof. Such forms include: solids, and in particular, tablets, filled capsules, powder and pellet forms; liquids, and in particular, aqueous or non-aqueous solutions, suspensions, emulsions, elixirs; and capsules filled with the same; all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The components of the compositions administered according to the methods of the present disclosure can be administered in a wide variety of oral and parenteral dosage forms. It will be obvious to those skilled in the art that the following dosage forms may comprise, in certain embodiments, as the active component, either a chemical compound of the present disclosure or a pharmaceutically acceptable salt of a chemical compound of the present disclosure.

For preparing pharmaceutical or nutraceutical compositions to be administered according to the methods of the present disclosure, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or encapsulating materials.

In powders, the carrier is a finely divided solid, which is in a mixture with the finely divided active component. In tablets, the active component is mixed with the carrier having the necessary binding capacity in suitable proportions and compacted in the shape and size desired.

In certain embodiments, powders and tablets administered according to methods of the present disclosure preferably may contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without additional carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include, but are not limited to, solutions, suspensions, and emulsions, for example, water or water-propylene glycol solutions. For example, parenteral injection liquid preparations can be formulated as solutions in aqueous polyethylene glycol solution. In certain embodiments, chemical compounds administered according to methods of the present disclosure may thus be formulated for parenteral administration (e.g., by injection, for example, bolus injection or continuous infusion) and may be presented in unit dose for administration in ampoules, pre-filled syringes, small-volume infusion, or in multi-dose containers with an added preservative. The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration in the mouth include, but are not limited to: lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example, with a dropper, pipette, or spray. The compositions may be provided in single or multi-dose form. In compositions intended for administration to the respiratory tract, including intranasal compositions, the compound will generally have a small particle size, for example, of the order of 5 microns or less. Such a particle size may be obtained by means known in the art, for example, by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself; or it can be the appropriate number of any of these in packaged form. Furthermore, dietary supplements are contemplated for use herein.

Tablets, capsules, and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of REMINGTON'S PHARMACEUTICAL SCIENCES (Mack Publishing Co., Easton, Pa.).

Routes of Administration

The compositions or blends may be administered by any route, including, but not limited to, oral, sublingual, buccal, ocular, pulmonary, rectal, and parenteral administration, or as an oral or nasal spray (e.g., inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection, or influsion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in the form of shaped articles, e.g. films or microcapsules.

Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entireties for all purposes.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

What is claimed is:

1. A delivery method for providing a prebiotic, a postbiotic, and an antioxidant, to a host organism, comprising the steps of:
(a) providing an xylo-oligosaccharide material including xylose units comprising formula (I): 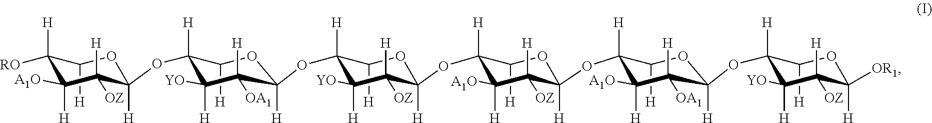
wherein R and R₁ are each independently selected from hydrogen or one or more xylose units,
A₁ is selected from hydrogen or acetyl,
Y is selected from the group consisting of hydrogen, arabinose (arabinosyl), galactose (galactosyl), ribose (ribosyl), mannose (mannosyl), glucuronic acid (glucuronosyl), and glucose (glucosyl),
Z is selected from the group consisting of hydrogen, glucuronic acid (glucuronosyl), galacturonic acid (galacturonosyl), and mannuronic acid (mannuronosyl),
optionally, Y and Z can be exchanged one for another, and
wherein either of Y or Z is further substituted on a sugar hydroxyl as a cinnamate ester, the positions of the phenyl group -meta, -para, -meta are each independently selected from hydrogen, hydroxyl, or methoxy, and
(b) administering the xylo-oligosaccharide material to the host in an amount effective to deliver acetic acid and/or a cinnamic acid derivative after enzymatic cleavage.

2. The delivery method of claim 1, wherein one or more xylose units of formula (I) is removed and replaced with a hydrogen atom.

3. The delivery method of claim 1 or 2, wherein the degree of polymerization (DP) is in a range of 2 to 10, or greater.

4. The delivery method of claim 1, wherein the degree of polymerization (DP) is greater than 10.

5. The delivery method of claim 1, wherein A₁ is acetyl in about one in six xylose units.

6. The delivery method of claim 1, wherein the cinnamate is present in an amount of about 1% by weight based on total solids.

7. The delivery method of claim 1, wherein the xylo-oligosaccharide is administered orally.

8. The delivery method of claim 1, wherein the host organism is a human.

9. A method for increasing Bifidobacteria or Lactobacillus in a human subject, comprising the steps of:
(a) providing an xylo-oligosaccharide material including xylose units comprising formula (I): 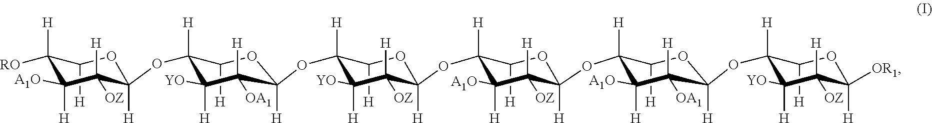
wherein R and R₁ are each independently selected from hydrogen or one or more xylose units,
A₁ is selected from hydrogen or acetyl,
Y is selected from the group consisting of hydrogen, arabinose (arabinosyl), galactose (galactosyl), ribose (ribosyl), mannose (mannosyl), glucuronic acid (glucuronosyl), and glucose (glucosyl),
Z is selected from the group consisting of hydrogen, glucuronic acid (glucuronosyl), galacturonic acid (galacturonosyl), and mannuronic acid (mannuronosyl),
optionally, Y and Z can be exchanged one for another, and
wherein either of Y or Z is further substituted on a sugar hydroxyl as a cinnamate ester, the positions of the phenyl group -meta, -para, -meta are each independently selected from hydrogen, hydroxyl, or methoxy, and
(b) orally administering the xylo-oligosaccharide material to the human subject in an amount effective to increase Bifidobacteria or Lactobacillus CFU counts in the gut or fecal matter of the human subject.

10. The method of claim 9, wherein the effective amount of xylo-oligosaccharide material is from about 1.0 g/day to about 3.0 g/day.

11. The method of claim 10, wherein the xylo-oligosaccharide material is administered for about 4 weeks to about 6 weeks.

12. The method of claim 11, wherein the Bifidobacteria CFU counts are increased by over 150% in the fecal matter.

13. The method of claim 11, wherein the Lactobacillus CFU counts are increased by about 35% in the fecal matter.

14. A method for increasing acetate in the gastrointestinal (GI) tract in a human subject, comprising the steps of:
(a) providing an xylo-oligosaccharide material including xylose units comprising formula (I): 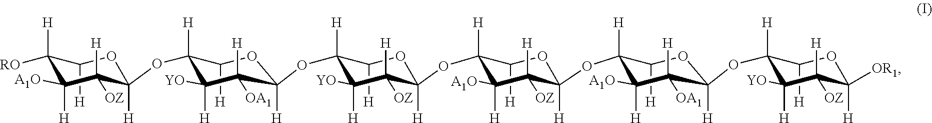
wherein R and R₁ are each independently selected from hydrogen or one or more xylose units,
A₁ is selected from hydrogen or acetyl,
Y is selected from the group consisting of hydrogen, arabinose (arabinosyl), galactose (galactosyl), ribose (ribosyl), mannose (mannosyl), glucuronic acid (glucuronosyl), and glucose (glucosyl),
Z is selected from the group consisting of hydrogen, glucuronic acid (glucuronosyl), galacturonic acid (galacturonosyl), and mannuronic acid (mannuronosyl),
optionally, Y and Z can be exchanged one for another, and
wherein either of Y or Z is further substituted on a sugar hydroxyl as a cinnamate ester, the positions of the phenyl group -meta, -para, -meta are each independently selected from hydrogen, hydroxyl, or methoxy, and
(b) orally administering the xylo-oligosaccharide material to the human subject in an amount effective to increase acetate in the gut or fecal matter.

15. The method of claim 14, wherein the effective amount of xylo-oligosaccharide material is from about 1.0 g/day to about 3.0 g/day.

16. The method of claim 15, wherein the xylo-oligosaccharide material is administered for about 3 days to about 7 days.
